(19) United States
(12) Patent Application Publication
(10) Pub. No.: US 2009/0093778 A1
(43) Pub. Date: Apr. 9, 2009

(54) DEVICE FOR ACTIVE TREATMENT AND REGENERATION OF TISSUES SUCH AS WOUNDS

(76) Inventor: PAUL SVEDMAN, Malmo (SE)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/062,930

(22) Filed: Apr. 4, 2008

Related U.S. Application Data
(63) Continuation of application No. 11/867,263, filed on Oct. 4, 2007.
(60) Provisional application No. 60/828,262, filed on Oct. 5, 2006.

(51) Int. Cl. A61F 13/02; A61M 1/00

(52) U.S. Cl. 604/290; 604/304; 604/313

(57) ABSTRACT

Hydrostatic pressure of aqueous solutions—supplied from reservoir under rate control through tube to port of airtightly applied open pore dressing pad—is eliminated by levelling reservoir placed on rest with pad. Dressing pad may overlie a tissue culturing scaffold. A drip chamber with angulated channel permits drops to fall freely and be counted. Injection port elastic membrane prevents air inlet to pad while suction is applied at port, permitting fluid given under rate control through membrane to distribute evenly in pad. A drainage port flange, wholly covered by an open grid, is described. Acute wound bleeding is detected by computer-controlled serial weighing of a movement-stabilized drainage fluid canister with warning of abnormal flow rate increase.

### CROSS-REFERENCE TO RELATED APPLICATIONS

This application claims priority to U.S. Provisional Application Ser. No. 60/828,262, filed Oct. 5, 2006, entitled “Device for Active Treatment and Regeneration of Tissues Such as Wounds,” which is incorporated herein by reference in its entirety.

### FIELD OF THE INVENTION

The present invention concerns a new device and method for treating tissues and open wounds and accomplishing directed tissue growth.

### BACKGROUND

Patients with open wounds, which cannot be sutured edge to edge, constitute a major health problem. Such wounds, which may include exposed muscle, tendon or bone, tend to become chronic. Poor blood supply, infection and dehydration are causative factors. Prolonged institutional care is usually required. Non-surgical treatment is usually followed by reconstructive surgery by means of skin grafts, composite tissue transfers or tissue regeneration.

Occlusive, pliable film dressings prevent dehydration and facilitate healing, but only in very superficial open wounds. In deeper wounds, “active” dressings permitting either supply of saline or therapeutic agents and/or suctioning of the wound surface improve healing by reducing tissue swelling, aiding contraction and stimulating healing. Continuously administered solutions influence the wound by diffusion processes. Therapeutic agents may constitute antibacterial substances for treating infection, enzymes for dissolution of non-viable material, growth factors or genes in tissue regeneration. Also cells may be supplied. In clean wounds, dressings combining fluid supply and drainage may facilitate adhesion of meshed skin grafts.

Wound suctioning by means of fluid-absorbing dextranomer beads (range of pressure depending on the degree of saturation of the beads; maximal suction −200 mmHg, that is 200 mmHg below atmospheric) has been reported. A cellular or fibrous (polyurethane, polyester etc) dressing pad with open pores with or without capillary activity, which may comprise layers with different qualities, and which is fitted with an impermeable cover sheet, was described in U.S. Pat. No. 4,382,441 and is incorporated herein by reference. Fluid is administered to the supply port of the said pad by pressure and/or drained from a drainage port by suction, and the supply tube contains a regulator valve. Fluid may thus be supplied to the pad 1) freely without or with suctioning, 2) in a rate-limited way combined with suction at the drainage port, or the fluid supply may be closed and the pad exposed only to suctioning. Using this invention, the wound was exposed continuously both to wetting and suctioning (−150 mmHg and −40 mmHg). Devices allowing intermittent or continuous fluid supply and/or suction drainage through a “spacer” comprising of a matted polyester fiber or polyurethane foam pad placed between the wound and the covering sheet were developed.

The dressing according to U.S. Pat. No. 4,382,441 may also be used for achieving tissue regeneration. An operational example in this said patent discloses that the open polymer dressing pad may be applied on a cell growth substrate (a collagen fiber scaffold, used in tissue regeneration, constitutes one such substrate). Also, growth substances may be supplied from said pad to the substrate, components of the substrate may be replaced and degradation products removed. A dressing according to the invention, used as a viability-maintaining device in vitro, i.e. a bioreactor, is described as an operational example in said patent.

### Functional Aspects of the Open Pore Dressings

During fluid supply and suctioning through an occlusively applied open pore dressing described in U.S. Pat. No. 4,382,441, the fluid volume forced by suctioning from capillaries and wounded tissue becomes added to that administered through the supply port. Functional aspects of this treatment are demonstrated in vitro in Examples 1-3. Example 1 suggests that in the presence of an unchanged flow resistance—located either in the fluid supply to the dressing or in the tissue—drainage fluid flow rate and suction pressure are directly correlated. The direct correlation between pressures in the drainage port and pad in a wide pressure range (Example 2) confirms previous findings (5). The dressing pad (Example 3) remains partially saturated even when treatment fluid is supplied at a rate of 7,200 mL/24 h. In vivo, treatment fluid is given at an approximate maximal rate of 2,400 mL/24 h, and the average rates by which tissue fluid form may range from 50 to 1,000 mL/24 h. The combined “maximal” in vivo fluid load of 3,400 mL/24 h (2,400+1,000 mL) thus clearly suggests a partially saturated state (3,400 mL/7,200 mL). The suctioning effect on the wound becomes abolished only when fluid or gas is allowed to enter the dressing freely or when the open pores have become clogged by biological material.

### Malfunction and Limitations of Open Pore Dressings Used for Combined Fluid Supply and Wound Suctioning on a Continuous or Intermittent Basis

Fluids may be supplied to the dressing either continuously by hydrostatic force from a drip stand, or by propulsion pump. Malfunction related to the hydrostatic pressure of the supplied fluid is at present neither recognized nor corrected for. Elevation of the fluid bag of a gravity drip for instance 68 cm or 136 cm above the dressing yields hydrostatic pressures of +50 and +100 mmHg respectively at the supply port. Dependency of the fluid bag relative to the dressing has the opposite effect. Pressure pumps expose the supply port to higher positive propulsive pressures, and may also include a significant positive or negative hydrostatic pressure component.

Viscous and particulate material or clots may predispose to gradual blocking of hydrophilic, capillary-active dressing pores, in particular near the drainage port. This will reduce the rate of fluid transport and also the suctioning force exerted on the wound surface. An eventually elevated hydrostatic pressure at the supply port becomes transmitted through the dressing pores to the blockage. Once the hydrostatic pressure exceeds the resistance in the dressing, a leak may result in overflow with wetting and soiling. If such blocking events are to be detected, complex electronic controls involving both supply and drainage would be required. A pressure sensor may reproducibly detect a pressure of +100 mmHg, but in a range extending towards +20 mmHg, the rate of false positive alarms will increase and reduce treatment practicality in a resource-demanding way. A simple and reproducible apparatus and method for eliminating hydrostatic pressure and achieving reliably a standardized combination of continuous therapeutic fluid supply with warning of impending dressing pore blockage is lacking, both in clinical wound treatment and tissue regeneration.

Known open pore dressings with supply and/or drainage ports (e.g., Principal AB, Malmö, Sweden; Kinetic Concepts, San Antonio, USA) lack means for reliable intermittent administration of saline or drug solution by injection during ongoing suctioning at the drainage port. Although local injection through the supply port can be accomplished with such devices, the need to leave the port open when fitting and removing a syringe or small volume fluid bag leads to immediate pressure equilibration between air and pad both before and after injection of the dosage. The first results in evacuation of the fluid representing the continuously supplied dosage from the pad, and the second in evacuation of the locally injected dose. A reliable apparatus and method for distributing treatment fluid intermittently to the wound tissue during continuous suctioning is thus lacking.

Bleeding from the wound during ongoing suctioning is an infrequent but at times life-threathening complication, which manifests itself by blood or plasma being sucked from the dressing. A simple means which may allow reproducible early detection of bleeding during ongoing suctioning is lacking.

### SUMMARY

In one or more embodiments, the use of positive hydrostatic or pump pressure as driving force for supplying fluid continuously to the open pore dressing is eliminated or minimized, and treatment fluid is sucked through the dressing pores by means of the suction pump used for distributing negative pressure to the wound. The placement of a fluid bag in bed at the level of the wound is impractical and prone to physical disturbance. Instead, the fluid reservoir (usually a pliable fluid bag) is placed on a support comprising a sloping or horizontal surface and the hydrostatic pressure is eliminated or minimized (i.e. to the level required for neutralizing flow resistance) by moving this said support vertically along a pole. This latter allows the fluid bag to be manually or automatically positioned level with the wounded tissue irrespectively of its height above the floor. The positioning may be facilitated using a horizontal level measuring device. In this apparatus and method, dependent on suctioning for function, one sensor which measures fluid supply rate will suffice for detecting malfunction.

A fluid administration set, intended to be used with the said fluid bag resting on said sloping or horizontal surface, comprising a drip chamber with angulated entry channel, which allows drops to fall freely, permits visual or automatic drop count. The said set may be fitted with a horizontal level meter and an injection port.

A supply port comprising of an elastic injection membrane is described. Intermittent doses of saline or drug solution can be administered against a resistance (cannula, syringe piston/wall contact, iv set rate-controlling device) from a syringe or fluid bag through this said elastic membrane to a dressing exposed to suctioning. This injection mode blocks air entry during connection and removal of the syringe, and allows the supplied fluid to distribute evenly throughout the dressing and over the wound surface as a result of vacuum and capillarity. Once fluid is detected visually in the suction tube a full intermittent dose has been given.

To prevent blocking the dressing pores at the drainage port by biological material, a suction port device is disclosed which contains an open grid means interposed between the whole area of said port and the open polymer dressing. This device maximally increases the area of dressing directly exposed to suctioning, augmenting the capacity of said port to eliminate particles and debris and increasing the duration of full function of the open pore dressing.

When using the dressing to supply nutrients for tissue regeneration, the rates of fluid transport and suctioning in the scaffold can be reduced to low levels to leave diffusional and cellular processes undisturbed. This is accomplished either by avoiding or minimizing hydrostatic pressure or pump head (to a level just sufficient to overcome both supply tube and open pore or tissue scaffold flow resistance) and applying concomitantly weak suction at the drainage port. In this latter situation more complex monitoring may be included.

An apparatus and method of allowing detection and warning of bleeding from a wound treated by suctioning comprising a computer connected with a scale, which measures serially the weight of the fluid sucked off the wound into an immobilized canister, and gives warning when the rate of fluid formation increases beyond that measured prior to the bleeding.

### BRIEF DESCRIPTION OF THE DRAWINGS

Embodiments of the present invention will now be described by way of example only and with reference to the accompanying drawings of which:

FIG. 1 shows a schematic view of a dressing system, according to an embodiment of the invention.

FIG. 2 shows two schematic cross-sections of another embodiment of the vertically movable rest including an alternative means for fixing said pole via a hook that can be clamped to the footboard of a bed.

FIG. 3 shows schematically an embodiment which includes motorized means for moving and fixing said platform at the desired level relative to the wounded tissue.

FIG. 4 shows a schematic cross-section of a fluid administration set for treating wounded tissue.

FIG. 5 shows an embodiment of said fluid administration set which includes a level meter and an injection port.

FIG. 6 shows a schematic cross-section of an open pore polymer dressing.

FIG. 7 shows a schematic cross-section of a non-protruding membrane injection port allowing intermittent supply of fluid during suctioning.

FIG. 8 shows a schematic perspective view of said port integrated in a dressing cover sheet used for self-assembly.

FIG. 9 shows a schematic cross-section of another embodiment of a membrane injection port invention.

FIG. 10 shows a schematic cross-section of a drainage port invention with open grid.

FIG. 11 shows a schematic cross-section of dressing having an open pore dressing overlying a scaffold used for tissue regeneration, according to another embodiment of the invention.

FIG. 12 shows a schematic cross-section of a device for detecting bleeding from the wound.

### DETAILED DESCRIPTION

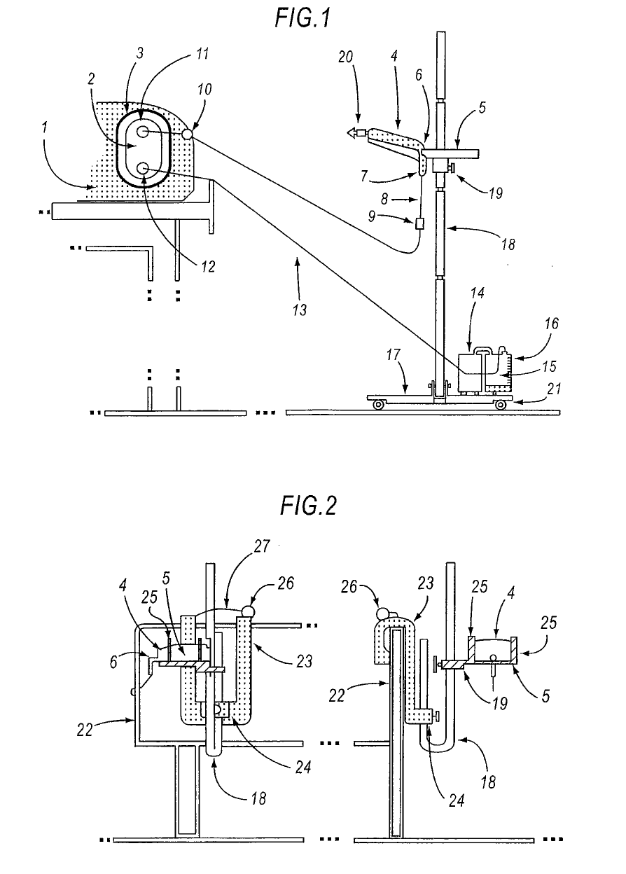

FIG. 1 shows one embodiment of the apparatus according to the invention. The dressing is sealed to the wounded tissue by means of a pliable polymer sheet. Treatment fluid contained in a pliable bag reservoir is connected to the supply port of the dressing by way of flexible tubing, while a suction pump is connected by tube to a drainage port. Fluid bag and horizontal level meter are placed on a rest which is movable along a pole in a vertical direction, and can be fixed in a position which is level with the wound. The pole is fixed on a base which holds a suction pump with canister.

More specifically, patient 1 is being treated with an open polymer dressing pad 2 sealed to the wounded tissue by means of a pliable polymer sheet 3. The means for accomplishing fluid flow comprises a pliable bag filled with treatment fluid 4 which is placed on rest 5. This bag connects by its outlet 6 to a drip chamber 7 which is followed by a flexible supply tube 8 comprising a fluid rate controlling device 9 and injection port 10 before connecting with the supply port 11 of the dressing 2. A rest 5 which is sloping in the area of the fluid bag is shown. The bag 4 is positioned on rest 5 with outlet 6 dependently, facilitating complete drainage and displacing eventual contained air upwards. The rest 5 can maintain fluid bag 4 in a flat or inclined plane in a range between 0° and 30°, allowing full fluid evacuation without, or with minimal, height difference between full and empty bag. A dressing drainage port 12 is connected by tube 13 to suction pump 14. Suction pump 14 and/or its fluid canister 15 may be connected to the line of suction anywhere—including on rest 5—where it may effectively drain fluid and prevent build-up of significant hydrostatic pressure within said tube 13. Canister 15 may be fitted with a scale 16 to allow determination of fluid volumes. The suction pump 14 may be fixed to the base 17, pole 18 or rest 5 by means of a screw, clamp or elastic strap. Rest 5 is movable in vertical directions along pole 18 and fixed by means of a clamp 19 in a position where the fluid surface in bag 4 and the supply port 11 of the dressing are level, i.e. the hydrostatic pressure at port 11 is at or near zero. This adjustment may be accomplished using a height indicator means 20 which may be connected with said rest 5. The means 20 may constitute for instance a horizontal level meter fixed either to a rewindable cord or to the proximal end of tube 8, or alternatively a telescopic pole or low energy red laser pointer connected with rest 5. The base 17 may be fitted with wheels 21. The rest 5, pole 18, and base 17 can for instance be made of aluminium, stainless steel or polycarbonate. Rest 5 can be moved along pole 18 manually, and locked at a suitable level by said clamp 19, or the pole may be height adjustable, constituted for instance of telescoping tubular sections which can be locked by screws at required length. The disclosed base 17 with pole 18 and rest 5 may be adapted for self-assembly.

FIG. 2 shows an embodiment of an apparatus according to the invention, which rids the rest 5 and its pole 18 of contact with the floor. In this device the footboard 22 of the bed is used as base. The construction minimizes the area of working-space occupied by the treatment devices and tubing. Rest 5 with U-shaped pole 18 is fastened to the footboard 22 by means of a hook-like structure 23 which may comprising elastic polymer or metal. The U-shape of pole 18 may allow positioning of rest 5 within a vertical range corresponding to the combined length of the two parallel vertical parts of pole 18, allowing the total length of pole 18 to be minimized. Immobilization of pole 18 is achieved by means of clamps 19 and 24. The fluid bag 4, placed on rest 5 with its outlet 6 dependent, is kept in place by vertical bars 25. An ultrasound distance sensor 26 is fixed to hook 23—for instance by means of an elastic or rewindable cord 27—and used for determining the horizontal level by measuring distances from wound and fluid bag to the roof. The device according to FIG. 2 may also be adapted for self-assembly. The straight pole 18 shown in FIG. 1 may fit in clamp 24 shown in FIG. 2 and used as an alternative to the u-shaped pole.

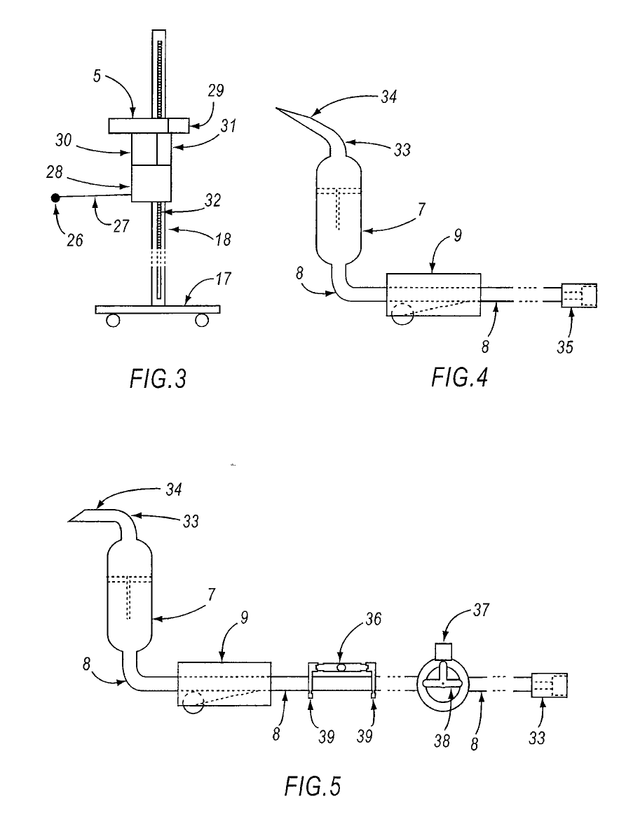

FIG. 3 shows an embodiment of an apparatus according to the invention, which may allow automatic movement and fixation of a horizontal rest 5 at a height along said pole 18 corresponding to the level of the wounded tissue at the press of a button. This may be accomplished by means of a unit comprising a computer 28 connected electronically with ultrasound sensors 26 and 29 and an electrical motor 30. The said motor 30 operates a cog-wheel 31 which meshes with another toothed part 32 extending along pole 18. Wire-based or hydraulic mechanisms may also be used. Once the said computer 28 receives for instance telemetric input on the distance from the wounded tissue to the roof from sensor 26 after activation by the therapist, computer 28 activates sensor 29 and moves rest 5 vertically to the same level. The said rest 5 may maximally be moved vertically in a range from 10 cm to 200 cm above floor level, which corresponds approximately to that of a lower leg wound on a sitting or standing patient and a wound on the head of a standing patient. The range of movement may also be restricted to fit patients lying or sitting in bed, with a range from 30 cm to 150 cm above floor level.

FIG. 4 shows an embodiment of an apparatus used according to the invention for administering treatment fluid from a fluid bag in a sloping or horizontal position. It comprises a transparent drip chamber 7 with an angulated entry channel 33 between the spike 34 and chamber 7. Said angulated entry channel 33 may be rigid or elastic, bendable to a chosen angle, and preferably made of polymer material. The chamber 7 is made of rigid polymer material. Tube 8 is made of pliable polymer material whose walls may be Luer format. The tube is fitted either with a roller clamp 9 or other mechanically or electronically operated device for controlling the flow rate—accomplished either by external compression of tube 8 or another known means of lumen reduction—and finally includes a connector 35 to the dressing supply port 11. The angulated entry channel 33 permits the chamber 7 to be approximately vertically positioned with the connected fluid bag placed on sloping or horizontal rest 5. This allows drops to fall freely in the chamber, permitting secure reading of the drip rate. Drip chambers of interest allow 40-80 drops per ml when exposed to negative pressures as high as 150 mmHg. Standard Luer format of tube 8, roller clamp 9 and connector 35 may be used but not obligatorily. The connector 35 to be fitted on the dressing port tube may alternatively constitute an elastic tube.

FIG. 5 shows a further embodiment of apparatus according to the invention to administer treatment fluid from a fluid bag. It differs from that described in FIG. 4 by including a horizontal level meter 36 and a tubular injection port connector 37. The latter may include tap 38 for directional control. The tubular connector 37 may be substituted by an elastic injection membrane. Said level meter 36, comprising for example a small gas bubble enclosed in a transparent glass tube filled with liquid fluid, is connected with the tube 8 for instance by arms 39 embracing tube 8. The level meter 36 should preferably be positioned near the drip chamber 7 to allow the meter 36 to be read while adjusting the height of the fluid bag 4 on rest 5 to match that of dressing 2. Connector 37 may be used for adding a drug dose to pad 2 during ongoing continuous supply and suctioning. To achieve optimal drip rate readability in chamber 7 of the embodiments shown in FIGS. 4 and 5, the combined angle of entry channel 33 and rest 5 for the fluid bag should be 90°.

Both the supply tube 8 and suction tube 13 may be thick-walled and/or corrugated at the inside to withstand kinking and compression, with inner diameter of approximately 3 mm. The suction tube 13 and canister 15 may be manufactured in pliable polymer materials. The sensor which measures fluid supply rate is suitably connected with an alarm.

In special situations, in particular associated with low-flow tissue culturing applications using pressure pumps, monitoring of volume rates of fluid supply and drainage may be included, as may pump head pressure and inadvertent gravity free flow. The propulsing force or head of the pressure pump should be just sufficient to achieve fluid flow. The pump should suitably be connected with a pressure sensor in the tube 8 or pad 2 to allow detection and adaption to a pump-related pressure disturbance. Computerized alarms concerning start/stop, occlusion, overflow and air-leak conditions may be applied. Known pressure and/or ultrasonic transducers or optical sensors may be used. A drip-sensing device may be attached to drip chamber 7. A timer-activated clamp may allow the fluid supply tube of a drip set to open and close at user-defined intervals, and a timer may control the start-stop function of the suction pump.

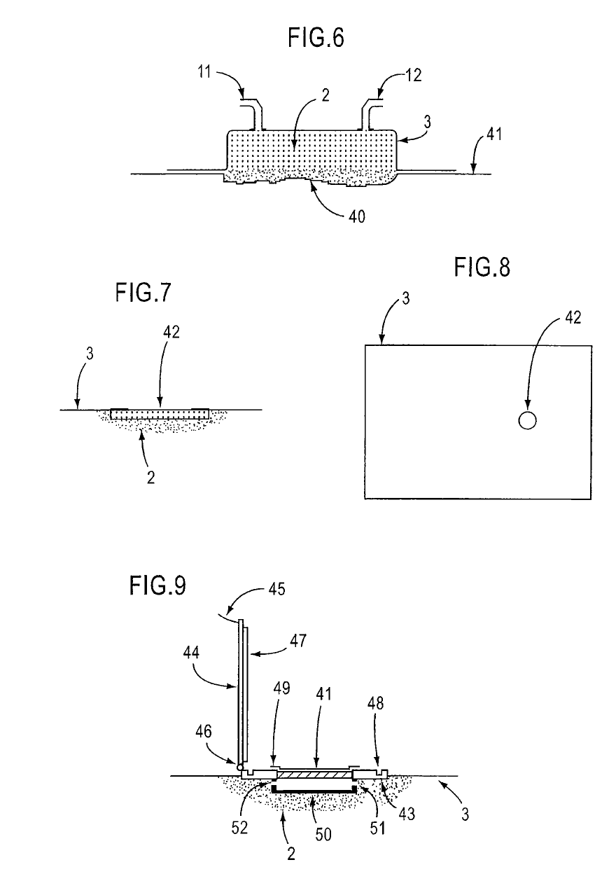

FIG. 6 shows an example of an open pore dressing with its pad 2 placed on the wound 40 and covered by a pliable, adhesive film or sheet 3, which is adhered to the adjacent skin 41. One supply port 11 and one drainage port 12 are adhered to the sheet 3 at a distance from each other. Each port is fitted with a tubular member, which allows the port to be connected to known supply or drainage tubes. Corresponding to these ports, apertures through the said sheet 3 allow access to pad 2 for fluid supply and suction drainage respectively. Flexible tubes are connected airtightly with the said ports 11 and 12, for instance by means of luer lock or elastic tube being forced over a conical rigid and tubular end-piece. The said sheet 3, ports 11,12 and tubing 8,13 provide a seal which allows the negative pressure within pad 2 and on the surface of wound 40 to be contained at a predefined level at least during operation of the suction pump. Pad 2 may comprise cell material with open pores or spatia like polyurethane or polyester foam or polyester fibers, and the latter may be matted. Pad 2 may include layers in which the pores and/or spatia have different dimensions. A thin dressing layer sandwiched between pad 2 and the wound surface may comprise knit or woven biofiber like cotton, wool or silk containing capillary functioning pores. This layer may be cut to fit sensitive areas of the wound where blood vessels and nerves are superficial or exposed. The sheet 3 may be fluid- or air-impermeable and is typically produced in polymer material (Minnesota Mining and Manufacturing, St. Paul, Minn. 55144). Pad, ports and tubes may be assembled either during fabrication, or bedside by the user. In a method according to the invention intended for treatment of wounds by means of the dressing shown in FIG. 6, the pressure at the supply port of the dressing is typically 0 mmHg, including correction for tube friction. This level of pressure is combined with suctioning at the drainage port ranging between −20 mmHg and −200 mmHg. This treatment may be applied intermittently or continuously for variable periods of time. The maximal suction applied under these circumstances is −760 mmHg. Fluid is supplied according to the invention at rates which may vary between 100 ml/24 h and 2,000 ml/24 h and loading doses for filling the dressing with drug solution may vary between 1 ml and 500 ml. This treatment may be undertaken on a continuous or intermittent basis.

An injection port device can be used for adminstering treatment fluid intermittently to the complete wound surface underneath dressing pad 2. This administration is always accomplished during ongoing suctioning through the drainage port 12. This apparatus, shown in FIG. 7, comprises an elastic membrane 42 which must have qualities which allow maintainment of occlusion after being perforated repeatedly by a needle. Membrane 42 may be connected adhesively to sheet 3, and the construction may or may not include a hole in said sheet 3 corresponding to the center of membrane 42. In connection with injection by needle through said membrane 42, the elastic qualities of membrane 42 should prevent formation of needle holes which could result in elimination of the vacuum in dressing pad 2. The membrane device may also be available as a separate unit with an adhesive rim at its circumference, the latter covered by removable protective paper, whereby said device can be applied adhesively around a hole in a dressing sheet 3 in a known manner. Finally, an adhesive, reusable and pliable lid may be placed on membrane 42 to maintain sterility between injections.

FIG. 8 shows an injection port apparatus according to FIG. 7 which is integrated in a pre-fabricated dressing sheet 3 intended for self-assembly, such that the dressing becomes complete by inclusion of dressing pad 2 and drainage port 12.

FIG. 9 exemplifies another embodiment of the said injection port apparatus, and comprising a rigid frame 43 which may be circular, and which is adhesively connected with the edges of a hole made in flexible sheet 3 covering the pad 2. The frame is likewise airtightly connected with elastic membrane 42. Frame 43 may be fitted with a lid 44, a handle 45, a joint 46 and a flange 47. When the device is not in use, lid 44 is closed over membrane 42. In this process, flange 47 enters a slit 48 in frame 43 to maintain secure occlusion. The membrane may optionally be protected by an adhesive tape patch 49 when not in use. Underneath the said membrane 42 is a rigid impermeable plate 50 which is connected by side walls 51 to said frame 43, and said walls 51 are fitted with apertures 52. The plate 50 may prevent the needle from inadvertently entering the wounded tissue in situations where the pad is thin. The membrane of the device according to FIG. 9 can suitably be manufactured in natural or synthetic rubber or elastic polymer including silicone. The frame, lid and plate structures may be manufactured in, for example, known, rigid polymer materials, and plate 50 may contain metal to prevent needle penetration. In operation during ongoing suctioning the lid 44 is opened by its handle 45, the piece of tape 49 is removed. A needle connected with a fluid-filled syringe is advanced through said membrane 42 while the membrane 42 is stabilized by holding lid 44. The fluid is slowly injected, allowing it to become distributed throughout the dressing by suction and capillary force. The supply of fluid may be terminated once fluid appears in the drainage tube. After injection the needle is removed and tape 49 and lid 44 are repositioned.

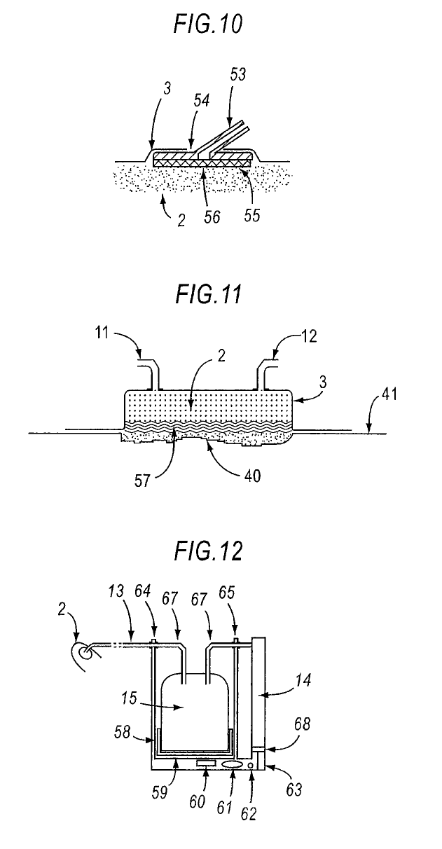

FIG. 10 shows an embodiment of an apparatus according to the invention comprising a drainage port which facilitates drainage of debris through a capillary-active dressing pad. It comprises a drainage port tube 53 with flange 54 sealed to pad 2 by means of an adhesive sheet 3. An open grid 55 adhered to the flange covers the entry to the opening 56 in the flange in order to avoid obstruction of dressing material against the edges of said opening 56. The grid is preferably manufactured of semi-flexible or rigid cells with open pores or spatia made of polymer materials, for instance polyester, polyurethane or steel wool, all typically less compressible to suction than dressing pad 2.

FIG. 11 shows an apparatus and method according to the invention comprising an open polymer pad 2 overlying a tissue culturing scaffold 57 used for regenerating skin tissue in a wound 40. The adhesive cover sheet 3, the supply port 11 and suction drainage port 12 are indicated. Depending on which type of tissue is to be regenerated, the scaffold may comprise biological and/or non-biological material. A biological scaffold may comprise collagen or dermis, hyaluronic acid or fibrin. When regenerating bone the scaffold may comprise bioactive ceramics or glass. Non-biological polymer fiber scaffolds may be biodegradable and comprise, for example, poly-glycolic acid polyester (PGA) or related substances. The optimal pore size of the scaffold may vary with the phase in the growth process. Passage by diffusion of all relevant nutrients and growth substances is obligatory throughout the regenerative process, and cell and vascular structures will have to be accommodated as they develop. The scaffold may thus include a range of more narrow pores which allow passage of molecules including peptides and proteins, as well as a range of wider pores allowing passage of cells, and this pore ratio may vary with the degree of tissue development. Antibacterial substances, analgesics, enzymes, growth factors, growth media and cells, including stem cells, fetal cells and genes, may be supplied.

In a method for accomplishing tissue regeneration in vivo or in vitro, see FIG. 11, the positive and negative pressures applied to pad 2 should be minimized in order to leave diffusional and cellular processes in the growth zone of the underlying tissue scaffold undisturbed. The forces governing the passage of fluid through the scaffold should be determined mainly by diffusion and minimal suction. This is accomplished in a controlled way by combining zero hydrostatic pressure or minimal pump head pressure in the dressing with at most weak suction at the drainage port. The hydrostatic pressure in the dressing can be 0 mmHg including compensation both for tube and open pore and/or tissue scaffold resistance. The suction pressures can range, for example, from −0 mmHg to −30 mmHg. The fluid supply rates may typically vary between 20 ml/24 h and 400 ml/24 h and loading doses for filling the dressing with drug solution may vary between 1 ml and 100 ml. This treatment may be undertaken on a continuous or intermittent basis. The pad 2 may be substituted for a tissue scaffold when the porosity of the scaffold allows passage of treatment fluid under flow and pressure conditions as defined above.

FIG. 12 shows an apparatus according to an embodiment of the invention for detecting bleeding from the wound during ongoing suctioning by a simple weighing technique. Canister 15 is immobilized in a tight-fitting receptacle 58 placed over load sensor 59, which in turn is connected to computer 60, display and control-panel 61, loudspeaker 62 and telemetric unit 63, all constructed according to the state of the art, and being part of the basal part 64 of the said apparatus. Elastical force or movement in suction tube 13, or movement in the pump 14 in operation, is prevented from being propagated to canister 15 by means of tubular buffer organs 64 and 65, each comprising a rigid and a pliable part. The rigid part constitutes in this example two closely fitting openings in the rigid receptacle wall 66. The pliable tubular part 67 is designed to further minimize movement and elastic force. The rigid part of each buffer organ may comprise polymer or metal. The pliable tubular part can be made of elastic polymer fitted with an inner discontinuous “skeleton” of rigid material to prevent collapse and occlusion. The pump 14 is additionally isolated with regard to vibration by means of elastic layer 68 placed between the base of the pump 14 and the basal part of the apparatus containing the computer and control means. This part of the apparatus can be made of metal to avoid vibration and increase stability.

The scale 59 may be operated by a load cell according to the state of the art. The computer 61 measures the weight of fluid in the canister 15 at pre-set intervals, and stores and displays the data using simple state of the art technology. The computer 61 first determines the baseline rate and variability of the therapeutic fluid formation over time based on measurements for instance at 2-5 min intervals. The smallest rate of fluid formed in addition to the therapeutic rate, which is to be considered as sign of a bleeding, is decided by the user and fed into the computer 61. The computer 61 then subtracts incoming rates from baseline serially and gives an audible, visual and possibly telemetric alarm once bleeding is detected. A bleeding in the wound may manifest itself 1) as a stepwise increase in liquid fluid weight 2) as a linear increase or 3) as an exponential increase. In a more advanced design, such patterns may also be identified and used by the computer as additional signs of bleeding. The computer can also warn of overflow of fluid in canister 15.

### EXAMPLE 1

Fluid flow rates in the dressing were studied in vitro as a function of the negative pressure applied at the suction port. The flow resistance was unchanged throughout.

### EXAMPLE 1

| | |
|---|---|
| Suction | Flow rate |
| mmHg (%) | ml/24 h (%) |

| | | |
|---|---|---|
| −50 | (−100) | 144 (100) |
| −100 | (−200) | 360 (250) |
| −200 | (−400) | 624 (433) |

Table I. Rate-limited fluid flow vs suction pressure in occlusively applied open polymer dressing with supply and drainage ports, studied in vitro.

The dressing comprising polyester fibers (11×13 cm) covered occlusively by polymer film and fitted with supply and drainage ports at opposing ends. The dressing was positioned horizontally.

The hydrostatic pressure at the supply port was 0 mmHg and the flow resistance in the supply was unchanged during the experiments. Fluid flow at the entry to the dressing and pump pressure were measured according to the state of the art.

### DISCUSSION

In this situation with unchanged resistance to entry of fluid into the porous dressing, fluid flow rate and suction pressure were close to linearly related.

### EXAMPLE 2

The negative pressure and degree of hydration in the dressing were studied in vitro as functions of the negative pressure at the drainage port.

### EXAMPLE 2

| | Step 1 | Step 2 | Step 2 | Step 3 |
|---|---|---|---|---|
| Fluid flow | 1440 | 1440 | 1440 | 1440 |
| rate | | | | |
| (ml/24 h) | | | | |
| Drainage | −15 | −50 | −100 | −200 |
| port gas | | | | |
| pressure | | | | |
| (mmHg) | | | | |
| Open pore | −13 | −46 | −93 | −180 |
| gas pressure | | | | |
| (mmHg) | | | | |
| Dressing | 52 | 40 | 50 | 35 |
| fluid | | | | |
| saturation | | | | |
| (Per cent) | | | | |

Table 2. In vitro assessment of pore gas pressure and fluid saturation during treatment according to the invention.

The dressing comprising polyester fibers (11×13 cm) covered occlusively by polymer film and fitted with supply and drainage ports at opposing ends. The dressing was positioned horizontally.

The hydrostatic pressure at the supply port was zero. Fluid flow was unchanged throughout the experiment. Pressure was measured in the drainage port and on the surface of the dressing pad. Dressing fluid saturation was measured by weighing, and calculated as percentage of the total saturable volume under influence of negative pressure as indicated.

### DISCUSSION

Suction pressures at the drainage port and within the pad were correlated over a pressure range of therapeutic interest. The dressing pad was partially saturated with fluid (mean: 44 percent, range: 35-52 percent). Clinically, a wound would thus be exposed dynamically to a combination of wetting and suction.

### EXAMPLE 3

The drainage capacity of a dressing exposed to fluid loading was studied in vitro.

### EXAMPLE 3

In vitro assessment of the drainage capacity of a dressing exposed to fluid loading.

The dressing comprising polyurethane foam (10×7.5 cm) covered occlusively by polymer film and fitted with supply and drainage ports at opposing ends. The dressing was positioned horizontally.

Fluid supply was increased from 20 drops/min to 100 drops/min in steps of 20 drops. The hydrostatic pressure at the supply port was zero. The suction pressure applied at the drainage port was −50 mm Hg. The thickness of the dressing was used as a measure of its compressed volume, and measured at each step. Dressing fluid saturation was assessed in the last step of the experiment, and determined as the percentage between the fluid contained in the dressing (assessed by weighing) and the total saturable volume assessed volumetrically during maximal suctioning.

### RESULT

The height of the dressing at each step of the experiment was compressed to approximately 7 mm. The dressing fluid saturation at 100 drops/min (equal to 7,200 mL/24 h) was 50/63 mL, and the maximal saturation thus 80%.

### DISCUSSION

This small format dressing remains partially saturated even when fluid is supplied at a rate as high as 7,200 mL/24 h. The results indicate that drainage capacity and hence a local suctioning effect is functional in a wide volume range at a pressure of −50 mmHg.

### CONCLUSION

In one embodiment, an apparatus for treating and regenerating tissues, covering a wound, combining liquid fluid supply and suction, comprises a pole, a rest, said rest being movable in vertical directions along said pole and having a clamp for securing said rest at a height corresponding to the height of the tissue, at least one fluid reservoir placed on said rest, connected to the tissue, and means for controlling the fluid supply and suction.

The rest can form an angle, for example, in the range 0-30° to the horizontal. The rest can be hinged, and immobilized in any angle from horizontal to vertical. The range of vertical movement of the rest can be, for example, approximately 10-200 cm, including for example 30-150 cm. A horizontal level meter can used for securing said rest at a height corresponding to the height of the tissue. The level meter such as a telescopic pointer, laser pointer or ultrasound sensor, can be connected with said rest directly or by means of a cord.

The fluid reservoir can comprise a pliable and flexible bag filled with treatment fluid. The fluid supply can be connected with the tissue by means of a tube.

The controlling means can include a drip chamber and a roller clamp connected with said tube. The controlling means include a drip chamber with an angulated spike connected with said tube. The controlling means can include a level meter connected with said tube. The controlling means can include an injection port connected with said tube. The means controlling the fluid supply can comprise an electronically operated valve. The means controlling the fluid supply can comprise a kink-resistant supply tube.

The apparatus can further comprise at least a drop-sensitive sensor for assessing the flow rate. The suction means can comprise a suction pump placed on the platform and connected to the tissue by means of a tube. The inner wall of said tube can be corrugated. The suction pump can be connected to a canister whose liquid fluid content can be determined by means of a scale or by weighing. The suction means can comprise a suction pump is placed on the floor. The suction means can comprise a pump is fixed to the pole by means of a clamp.

The pole is, for example, u-shaped and fixed by means of a clamp to a hook which can be fastened to the footboard of a bed. The pole can be straight and fixed to a base. The pole can comprise telescoping parts which can be locked in position by means of screws, clamps or by a hydraulic mechanism.

The apparatus can farther comprise a motor which moves said rest in a vertical direction and which is operable by means of a computer. The apparatus can also comprise ultrasound level meters, one fixed and one movable, and both connected to said computer. The apparatus can further comprise a pump used for administering the fluid supply. The pressure head of the said pump can be monitored by means of a sensor in the supply tube. The fluid flow can be controlled by means of timer activated clamps.

In one embodiment, apparatus for treating and regenerating tissues allowing administration of a restricted amount of fluid to the supply port of an occlusively applied porous dressing pad during exposure of said pad to continuous suctioning through a separate drainage port, comprising a restricting means preventing free fluid flow, a means to prevent ingress of air through said supply port in connection with said fluid administration, and a drainage port.

The supply port can comprise of an injection membrane airtightly connected with a polymer sheet. The supply port can comprise a plate at the side of the dressing pad which can prevent a needle used for injecting treatment fluid through said membrane from penetrating into the dressing and to the wound.

The apparatus can further comprise a roller clamp that provides additional restricting means. The friction between piston and syringe wall can provide additional restriction means.

In another embodiment, a method for treating and regenerating tissues allowing administration of a restricted amount of saline or drug solution to the supply port of an occlusively applied porous dressing pad during exposure of said pad to continuous suctioning, comprises: applying continuous suction to the drainage port in the range between 30 and 200 mmHg; applying an injection needle airtightly to a syringe or fluid bag filled with saline or drug solution; avoiding a fluid bag hydrostatic load; perforating said supply port elastic injection membrane with the needle during ongoing suction at the drainage port of said dressing pad; injecting the content of the syringe into the dressing pad in 1-5 minutes during ongoing suctioning at said drainage port; stopping the injection once injected fluid becomes visible through the suction tube wall as it exits the drainage port of the dressing pad; and withdrawing the said needle from the elastic membrane.

In yet another embodiment, a method for non-regenerative tissue treatment by means of combined fluid supply and suction drainage to a porous dressing, comprises: eliminating hydrostatic pressure in the fluid supply port by positioning the fluid bag at the level required for neutralizing supply tube flow resistance; maintaining the tissue hydrostatic pressure at the supply port at 0 mmHg; maintaining the fluid flow in a range between 100 ml/24/h and 2,400 ml/24 h; providing a seal which allows negative pressure to be distributed over the tissue and to be maintained at a predetermined level at least during operation of the suction;

maintaining the suction normally in a range between −20 mmHg and −200 mmHg, maximally −760 mmHg; utilizing loading doses in the range between 1 ml and 500 ml; and applying steps a-f continuously or intermittently.

In yet another embodiment, a method for regenerative treatment by means of combined fluid supply and suction drainage to a tissue scaffold, comprises: eliminating hydrostatic pressure by positioning the fluid bag at a level just sufficient to overcome both supply tube and/or open pore scaffold flow resistance; maintaining the fluid flow in the range between 20 ml/24/h and 400 ml/24 h; providing a seal which allows negative pressure to be distributed over the tissue and to be maintained at a predetermined level at least during operation of the suction; maintaining the suction in the range between −0 mmHg and −30 mmHg; utilizing loading doses in the range between 1 ml and 100 ml; and applying steps a-e continuously or intermittently.

In yet another embodiment, a method for regenerative treatment allowing artificial circulation to a tissue scaffold, comprises: eliminating hydrostatic pressure by positioning the fluid bag at a level just sufficient to overcome supply tube, porous pad and/or scaffold flow resistance; controlling fluid supply rate by interposing a pump in the supply line; monitoring the pressure head in the supply port; monitoring the pressure in the porous pad or scaffold; maintaining the tissue hydrostatic pressure at the supply port at 0 mmHg; maintaining the fluid flow in the range between 20 ml/24/h and 400 ml/24 h; providing a seal which allows negative pressure to be distributed over the tissue and to be maintained at a predetermined level at least during operation of the suction; maintaining the suction in the range between −0 mmHg and −30 mmHg; utilizing loading doses in the range between 1 ml and 100 ml; and applying steps a-i continuously or intermittently.

In another embodiment, an apparatus for treating and regenerating tissues by means of an occlusively applied dressing pad, comprises a drainage port with means to counteract occlusion of the underlying open pores of the pad when said pad is exposed to continuous suction. The drainage port means can comprise an open grid consisting of interconnected or separate units which form a pattern covering the whole underside of the port abutting the dressing pad. The grid can include the opening in the flange.

In another embodiment, an apparatus for detecting bleeding from a wound during continuous suctioning treatment comprises a receptacle, a scale, a canister, movement buffer organs, a computer, visual display, audible alarm and telemetry.

In another embodiments, a method for detecting bleeding from a wound during continuous suctioning treatment, comprises: determining the baseline rate and variability of therapeutic fluid formation over time based on measurements of net weights of fluid in the canister at 2-5 min intervals; determining of the minimal rate of fluid formed in addition to said baseline rate which is to be considered as a sign of bleeding, and feeding this information to the computer; making the computer subtract incoming rates of fluid formation from baseline serially, and giving an audible, visual and telemetric alarm once bleeding is detected.

1-20. (canceled)

21. An apparatus, comprising:
a wound dressing;
a membrane having a first side and a second side opposite the first side, the membrane defining an aperture, the membrane configured to be disposed on the wound dressing such that the first side of the membrane is disposed between the wound dressing and the second side of the membrane; and
a port having a collar portion and a flange portion, the collar portion having a first side with an adhesive coating and a second side opposite the first side of the collar portion, the collar portion defining a plane substantially parallel to the second side of the membrane, the first side of the collar portion configured to adhere to the second side of the membrane to produce a seal between the port and the membrane without connecting the second side of the collar portion of the port to the first side of the membrane,
the flange portion of the port defining a lumen configured to be in fluid communication with the aperture of the membrane and in fluid communication with the wound dressing, at least of portion of the flange portion of the port having a longitudinal axis substantially transverse to the plane defined by the collar portion.

22. The apparatus of claim 21, wherein the wound dressing is configured to contact a wound surface, the apparatus further comprising:
a suction source; and
a tube having a proximal end portion configured to be coupled to the suction source and a distal end portion configured to be coupled the flange portion of the port,
the suction source configured to produce negative pressure within the wound dressing and on the wound surface when the wound dressing, the membrane and the port are assembled with respect to the wound surface.

23. The apparatus of claim 21, further comprising a second port having a collar portion and a flange portion, the collar portion of the second port configured to adhere to the second side of the membrane, the flange portion of the second port defining a lumen configured to be in fluid communication with a second aperture defined by the membrane and in fluid communication with the wound dressing.

24. The apparatus of claim 21, further comprising:
a second port having a collar portion and a flange portion, the collar portion of the second port configured to adhere to the second side of the membrane, the flange portion of the second port defining a lumen configured to be in fluid communication with a second aperture defined by the membrane and in fluid communication with the wound dressing; and
a tube having a proximal end portion and a distal end portion, the proximal end portion configured to be coupled to a fluid-supply vessel and the distal end portion configured to be coupled to the flange portion of the second port.

25. The apparatus of claim 21, wherein the port is unitarily formed.

26. The apparatus of claim 21, further comprising:
a tube having a proximal end portion and a distal end portion, the tube defining a lumen, the distal end portion of the tube configured to fit over the flange portion of the port to produce an airtight connection between the flange portion of the port and the tube.

27. The apparatus of claim 21, further comprising:
a tube having a proximal end portion and a distal end portion, the tube defining a lumen; and
a releasably lockable connector configured to produce an airtight connection between the flange portion of the port and the tube.

28. An apparatus, comprising:
a fluid permeable dressing;
a membrane having a first side with an adhesive and a second side opposite the first side, the first side of the membrane configured to adhere to and extend over the fluid permeable dressing, the membrane defining an aperture;
a unitarily-formed port having a collar portion and a flange portion, the collar portion having a first side with an adhesive coating and a second side opposite the first side of the port, the first side of the collar portion configured to adhere to the second side of the membrane, the flange portion of the port defining a lumen configured to be in fluid communication with the aperture of the membrane and in fluid communication with the wound dressing; and
a tube having a proximal end portion and a distal end portion, the distal end portion of the tube configured to be removably coupled to the flange portion of the port.

29. The apparatus of claim 28, further comprising a releasably lockable connector configured to couple the distal end portion of the tube to the flange portion of the port.

30. The apparatus of claim 28, wherein the fluid permeable dressing is configured to contact a wound surface, the apparatus further comprising:
a suction source configured to produce negative pressure within the fluid permeable dressing and on the wound surface when the fluid permeable dressing, the membrane and the port are assembled with respect to the wound surface.

31. The apparatus of claim 28, further comprising a second port having a collar portion and a flange portion, the collar portion of the second port configured to adhere to the second side of the membrane, the flange portion of the second port defining a lumen configured to be in fluid communication with a second aperture defined by the membrane and in fluid communication with the fluid permeable dressing.

32. The apparatus of claim 28, further comprising:
a second port having a collar portion and a flange portion, the collar portion of the second port configured to adhere to the second side of the membrane, the flange portion of the second port defining a lumen configured to be in fluid communication with a second aperture defined by the membrane and in fluid communication with the fluid permeable dressing; and
a second tube having a proximal end portion and a distal end portion, the proximal end portion of the second tube configured to be coupled to a fluid-supply vessel and the distal end portion of the second tube configured to be coupled to the flange portion of the second port.

33. The apparatus of claim 28, wherein the distal end portion of the tube is configured to fit over the flange portion of the port to produce an airtight connection between the flange portion of the port and the tube.

34. An apparatus, comprising:
a wound dressing having a wound surface defining a perimeter;
a membrane having an upper side and a lower side opposite the upper side, the membrane defining an aperture and defining a perimeter, the membrane configured to envelope the wound dressing such that the lower side of the membrane contacts the wound dressing and the perimeter of the member is outside the perimeter of the wound dressing;
a port having a first portion and a second portion, the first portion being substantially planar and defining an aperture, the second portion being substantially non-planar, the first portion having a first side with an adhesive coating configured to adhere to the upper side of the membrane such that the aperture of the first portion is substantially aligned with the aperture of the membrane, the second portion defining a lumen; and
a tube having a proximal end portion and a distal end portion configured to be removably coupled to the second portion of the port.

35. The apparatus of claim 34, wherein the first portion of the port has a second side opposite the first side, the second side of the first portion of the port being uncovered and without an adhesive when the wound dressing, the membrane and the port are assembled.

36. The apparatus of claim 34, further comprising a releasably lockable connector configured to couple the distal end portion of the tube to the flange portion of the port.

37. A method, comprising:
disposing a first side of a fluid permeable dressing in contact with a wound surface, a second side of the fluid permeable dressing being opposite the first side;
adhering an adhesive sheet to the second side of the fluid permeable dressing via an adhesive coating on a first side of the adhesive sheet, a second side of the adhesive sheet being opposite the first side of the adhesive sheet; and
adhering a first side of a collar portion of a port to the second side of the adhesive sheet via an adhesive coating on the first side of the collar portion of the port; and
attaching a distal end of a tube to a flange portion of the port after the adhering the first side of the collar portion to produce an airtight connection between the tube and the flange portion of the port.

38. The method of claim 37, wherein the adhering the first side of the collar portion of the port is sufficient to define a seal between the port and the adhesive sheet without a second adhesive sheet.

39. The method of claim 37, further comprising:
the collar portion of the port defining a plane substantially parallel to the second side of the fluid permeable dressing, the attaching being outside the plane defined by the collar portion of the port.

40. The method of claim 37, further comprising:
adhering a first side of a collar portion of a second port to the second side of the adhesive sheet via an adhesive coating on the first side of the connection portion of the second port; and
introducing a fluid to the wound surface by attaching a distal end of a tube to a flange portion of the port and attaching a proximal end of the tube to a fluid-supply vessel.
